(19) United States
(12) Patent Application Publication
(10) Pub. No.: US 2023/0405056 A1
(43) Pub. Date: Dec. 21, 2023

(54) Extraction Method of Natural Crocodile Oil and Extracted Crocodile Oil

(71) Applicant: Lixi Xie, Zhuhai City (CN)

(72) Inventor: Lixi Xie, Zhuhai City (CN)

(21) Appl. No.: 18/196,018

(22) Filed: May 11, 2023

(30) Foreign Application Priority Data
Jun. 16, 2022 (CN) 202210681323.6

(51) Int. Cl. A61K 35/58; C11B 1/02; C11B 3/00; A61K 36/28; A61K 36/736; A61K 36/324; A61K 36/53; A61K 36/23; A61K 36/8962

(52) U.S. Cl. CPC A61K 35/58; C11B 1/02; C11B 3/008; A61K 36/28; A61K 36/736; A61K 36/324; A61K 36/53; A61K 36/23; A61K 36/8962

(57) ABSTRACT

The present disclosure provides an extraction method of natural crocodile oil. The method improves the traditional ancient method of making oil, and includes: mixing crocodile fat with a traditional Chinese medicine bag and standing, then taking out the fat, and sequentially pulping and filtering. By using the above method, any extracts are not added without decolorization, the effective ingredients in the crocodile oil can be maintained to the greatest extent to the greatest extent, and therefore the method of the present disclosure is suitable for large-scale production. The present disclosure further provides the crocodile oil obtained by the extraction method. The crocodile oil is glittering and translucent, has no fishy smell, and can be preserved for a long time.

### CROSS REFERENCE TO RELATED APPLICATION

This application claims the benefit of and takes priority from Chinese Patent Application Serial No. 202210681323.6 filed on Jun. 16, 2022, the contents of which are herein incorporated by reference.

### TECHNICAL FIELD

The present disclosure relates to the technical field of animal oil fat extraction, particularly to an extraction method of natural crocodile oil, and crocodile oil obtained by the extraction method.

### BACKGROUND

Crocodile oil is oil extracted from crocodiles, and mainly comprises the following components: unsaturated fatty acids, trace elements and other nutrient substances. The physicochemical properties of the crocodile oil are particularly similar to those of human body fat, and therefore the crocodile oil has good permeability and very easily accepted and absorbed by human body. The crocodile oil functions to comprehensively regulate the metabolic activity of skin cells, promote the immunologic function, efficient permeability and multi-layer support of the skin, moisturize and comprehensively balance nutrition, thereby realizing the effects of moisturizing, wrinkle removing, repairing, whitening, aging resisting and the like.

At present, there are few of extraction and refine methods and processes for crocodile oil, and most of them refer to fish oil extraction methods. The crocodile oil extraction methods mainly include an organic solvent extraction method, an alkali hydrolysis method, a vacuum distillation method, a supercritical CO₂ extraction method, a dry boiling method, etc.

Where, the organic solvent extraction method has the advantages that different components are separated by selecting different reagents according to an extraction principle. The organic solvent extraction method has the disadvantages that this method is low in extraction efficiency and suitable for research in a lab rather than large-scale production, and many organic reagents are left.

The alkali hydrolysis method has the advantages that proteins in fat tissues are hydrolyzed by using a diluted alkali aqueous solution so that oil binding to the proteins is dissociated, which is a common method in a fish oil extraction industry. The alkali hydrolysis method has the characteristics of high oil yield, light product color, few free fatty acid content and the like. The alkali hydrolysis method has the disadvantages that the hydrolysis of fish oil can be saponified during the treatment so as to damage the active ingredients of fish oil while leading to increase in pH of fish oil.

The supercritical CO₂ extraction method has the advantages that since carbon dioxide has a supercritical temperature of 31.1 degrees and a critical pressure of 7.374 MPa, the operation temperature is relatively low, and carbon dioxide is an inert gas and can prevent the oxidization of fish oil. The supercritical CO₂ extraction method has the disadvantages that since the crocodile fat is wrapped in lots of collagen fibers and adipocytes, many ingredients cannot be extracted, with low extraction rate (about 30%), long extraction time and large energy consumption.

The dry boiling extraction method has the advantages of high extraction efficiency, short extraction time and simple process. The dry boiling extraction method has the disadvantages of high extraction temperature, easy damage of unsaturated fatty acid, dark color and heavy fishy smell.

The biological enzymolysis method has the advantages that protein components such as collagen fibers and adipocyte membranes are cleaved through specific enzymes, so that fish oil is fully precipitated; due to its relatively low action temperature and short enzyme action time, the enzyme can prevent oxidization of fish oil during the extraction; furthermore, the enzyme has high specificity and do not damage the ingredients of fish oil during the cracking, the extracted fish oil is clear and transparent without decolorization and has complete ingredient composition, its activity can be maintained to the greatest extent, so this method is currently the best extraction method, and professionally improved enzymes can achieve an extraction rate of over 80%, with high efficiency. The biological enzymolysis method has the disadvantages that due to high specificity of enzymes, the action sites of enzymes are needed to be screened and improved for different species, so research and development period is long, and enzymes are expensive in price and are only used once during the extraction, with high cost, enzymes are often treated as expensive samples, and most of products are used for high-end markets. Furthermore, the crocodile fat tissue treated by enzymolysis is a mixture of oil, tissue fragments and water, and is difficultly separated when production on large scale.

There are few researches on decolorization of crocodile oil, which often refer to decolorization methods of fish oil. In most cases, physical adsorption and chemical reagent decolorization are adopted.

Where, in the physical decolorization method, activated carbon, activated clay, attapulgite, and diatomaceous earth are often used for adsorption and decolorization. Activated carbon, activated clay are used in patent CN201310451356.2 for decolorization of crocodile oil. The physical decolorization method has the advantages that the physical adsorption method is used, which is safe. The physical decolorization method has the disadvantages that fish oil needs to be heated during the adsorption and decolorization, which can lead to oxidation, increased fishy smell and decreased activity of fish oil, and the treatment requirement of the adsorbent is high, or else impurities therein will also contaminate the fish oil.

At present, there are very few researches on a deodorization process of crocodile oil, which mainly refer to deodorization methods of fish oil, including physical deodorization, chemical deodorization, and biological deodorization.

Where, physical deodorization mainly adopts a physical adsorption principle, which often uses activated carbon, silica gel, diatomaceous earth and other materials. The physical deodorization has the advantages that some fishy smells can be removed without bringing impurities. The physical deodorization has the disadvantages that the removal rate of fishy smell is low; heating is needed during the adsorption to damage the ingredients of crocodile oil, whereas fishy smell is easily returned back after cooling; and new impurities are often brought into unqualified materials, so as to damage the fish oil. In the patent CN102304419A for crocodile oil deodorization, a method for deodorization by passing through a silica gel-traditional Chinese medicine column.

The chemical deodorization mainly uses a method of adding acids and alkali and adding organic reagents to remove fishy smells. The chemical deodorization has the advantages that the removal effect of fishy smells is better than that of the physical adsorption and deodorization. The chemical deodorization has the disadvantages that addition of acids and alkali and organic reagents damage the ingredients of crocodile oil to a certain extent, and the heating process can cause the degradation of crocodile oil is and saponified to reduce the activity, and the organic reagents can also be left to a certain extent. In the patent CN201310451356.2 for deodorization of crocodile oil, first, acid was added and then heating was performed, followed by adding alkali and then heating, and finally the remaining saponified substances were washed away with hot water to remove fishy smells.

The biological method for deodorization is that fishy ingredients in crocodile oil are removed by targeted consumption of microorganisms. The biological method has the advantages that the temperature is low, the processing conditions are mild, the time is short, and the ingredients of fish oil are not damaged, and the fishy smell is completely removed without repetition. The biological method has the disadvantages that the process requirements are very high, the treatment process requires strict prevention of contamination by other microorganisms, the screening and specificity improvement of microorganisms have high technical difficulty, and great research and development difficulty, the requirements on production environment of a factory are extremely high, and factories that meet GMP certification generally meet the basic conditions. The biological method for deodorization is an emerging technology, which is still in the forefront of research, and has great prospects.

In view of this, the present disclosure is put forward.

### SUMMARY

In order to overcome the above technical problem, it is needed to improve the extraction process of crocodile oil so that the extraction process is simplified to the greatest extent, and the active components therein are maintained maximally.

The first objective of the present disclosure is to provide an extraction method of natural crocodile oil. The method improves the traditional ancient method of making oil without addition of any extracts and decolorization, maintains effective components in crocodile oil to the greatest extent, and is suitable for large-scale production.

The second objective of the present disclosure is to provide crocodile oil extracted by the method. The obtained oil has no fishy smell and a crystal and transparent oil body, and is suitable for long-time storage.

To achieve the above objectives, the technical solution of the present disclosure is as follows:

The present disclosure relates to an extraction method of natural crocodile oil, the method comprising the following steps:

(1) washing crocodile fat, smashing, adding the smashed crocodile fat into a traditional Chinese medicine bag, and then standing; and

Preferably, the standing time is 30-50 hours.

Preferably, the traditional Chinese medicine bag comprises the following components: calendula, cherry blossom, frankincense, patchouli, centella asiatica, and allium macrostemon.

Preferably, a mass ratio of the calendula to the cherry blossom to the frankincense to the patchouli to the centella asiatica to the allium macrostemon is 1:1:1:1:1:1.

Preferably, a mass ratio of the crocodile fat to the traditional Chinese medicine bag is (1500-2500): (3-4).

(2) taking out the fat and then pulping to obtain a muddy material, filtering the muddy material, and then collecting filtrate to obtain crocodile oil.

Preferably, the pore diameter of the filter mesh is 150-300 meshes.

Preferably, step (1) and step (2) are carried out at an ambient temperature of 15-25° C.

The present disclosure further relates to crocodile oil extracted by the method.

The present disclosure has the beneficial effects:

The present disclosure provides an extraction method of natural crocodile oil. The method improves the traditional ancient method of making oil, including mixing crocodile fat with a traditional Chinese medicine bag and standing, then taking out the fat, and sequentially pulping and filtering. In the method, there is no need to add any extracts without decoloring. The effective ingredients in the crocodile oil can be maintained to the greatest extent to the greatest extent, and is suitable for large-scale production.

The present disclosure further provides the crocodile oil obtained by the extraction method. The crocodile oil is glittering and translucent, has no fishy smell, and can be preserved for a long time.

### BRIEF DESCRIPTION OF THE DRAWINGS

FIG. 1 is a picture of crocodile oil extracted in example 1 at room temperature of 20° C.

FIG. 2 is an eczema picture of an infant before crocodile oil is used.

FIG. 3 is an eczema picture of an infant after crocodile oil is used for one month.

FIG. 4 is an eczema picture of an infant after crocodile oil is used for one and a half months.

### DETAILED DESCRIPTION OF THE EMBODIMENTS

To make the purpose, technical solution and advantages of the present disclosure more clear, the technical solution of the present disclosure will be described in detail below. Obviously, the described embodiments are only some embodiments of the present disclosure but not all the embodiments. Based on the embodiments of the present disclosure, all other embodiments made by persons of ordinary skill in the art without creative efforts are included within the scope of protection of the present disclosure.

The present disclosure relates to an extraction method of natural crocodile oil, the method comprising the following steps:

(1) washing crocodile fat, smashing, adding the smashed crocodile fat into a traditional Chinese medicine bag, and then standing; and

In one embodiment of the present disclosure, the standing time is 30-50 hours. By standing, the drug components in the traditional Chinese bag enter crocodile oil, so as to promote the edible and external effects of the crocodile oil.

In one embodiment of the present disclosure, the traditional Chinese medicine bag comprises the following components: calendula, cherry blossom, frankincense, patchouli, centella asiatica, and allium macrostemon.

The effects of the above traditional Chinese medicine components are as follows:

(Calendula)

Calendula, also known as Calendula officinalis, gold calendula, helichrysum arenarium, hops, asphodel, marigold, etc., the flowers and leaves of calendula have anti-inflammatory and antibacterial effects, the root of calendula is capable of promoting qi and blood circulation, and the flowers of calendula are capable of cooling blood and stopping bleeding. The calendula has a beauty effect that cannot be ignored, which can well moisturize dry skin.

The Calendula officinalis has a light and flat taste, and its flowers and leaves have anti-inflammatory and antibacterial effects. The root of Calendula officinalis is capable of promoting qi and blood circulation, and the flowers of Calendula officinalis are capable of cooling blood and stopping bleeding. In Europe, it can be used externally for various inflammations of the skin and mucous membranes, and orally administrated for the treatment of various inflammations and ulcers. Fresh flowers can be put in salads to be eaten. The calendula is rich in flavonoid active ingredients which have anti-oxidant, anti-vascular proliferation, anti-inflammatory, anti-allergic, and anti-viral effects.

The calendula contains mineral phosphorus, vitamin C etc., and its main utilization part is petals. Drinking calendula tea can cause sweating, diuresis, and clearing dampness and heat. Furthermore, its flowers can be made into salads or used for cooking to enhance color, aroma, and flavor. The calendula is also a powerful herb, which can be mainly used to treat skin diseases and wounds. Externally using the calendula has anti-inflammatory, bactericidal, anti-mold, astringent and anti-ulcerative effects, and alleviates sunburn and scalding. If being used for steaming, herbal baths, or hand and foot baths, the calendula can play a role in promoting the cleanliness and softness of the skin.

The functions of the calendula are as follows:

1. Calendula has a super-strong healing ability, can sterilize and astringe wounds, treat inflammation, acne, and rough pores, and prevent the formation of scars.

2. Calendula, is also known as Calendula officinalis and has a light and flat taste, and its flowers and leaves have anti-inflammatory and antibacterial effects. The root of calendula officinalis is capable of promoting qi and blood circulation, and the flowers of Calendula officinalis are capable of cooling blood and stopping bleeding. In Europe, it can be used externally for various inflammations of the skin and mucous membranes, and orally administrated for the treatment of various inflammations and ulcers. Due to its beauty effect, the calendula is used as one of the functional ingredients of cosmetics brands such as Cher's and Swiss magic care magic care.

3. Calendula can moisturize and dry lips and promote skin metabolism. Especially for dry skin, it has a good moisturizing effect.

4. Calendula can calm the skin and improve sensitive skin texture, and has the effect of repairing scars.

The calendula tea is brewed from a tablespoon of dried petals and simmered for about 10 minutes. The golden tea is set off against the floating golden petals, with a refreshing fragrance. It tastes sweet and slightly bitter when consumed, and can be mixed with honey. Drinking calendula tea when catching a cold can help reduce fever and cool down the fire. The calendula tea also has the effects of relieving spasms and promoting digestion, making it extremely suitable for patients with digestive system ulcers. In addition, it can also promote blood circulation, alleviate alcoholism, and is beneficial for nourishing the liver.

(Cherry blossom)

Cherry blossom for medication: cherry blossom peel and wood contain 5-glucoside and 5-sakurarin of gentisic acid and cherry glycoside, the wood contains d-catechin; stems and leaves contain quercetin 3-galactoside; tender leaves contain coumarin, trans-o-hydroxycinnamic acid glucoside, and cyanoside; seed kernels contain 32% fatty oil, mainly containing a-tungic acid and sitosterol, and are used for cough and fever or other symptoms.

Cherry blossom for skin care: the cherry blossom has good pore-shrinking and oil-balancing effects and contains rich natural vitamins A, B, and E, flavonoids contained in cherry blossom leaves also have the efficacy of beautifying, strengthening mucous membranes, and promoting sugar metabolism.

The cherry blossom extract contains cherry blossom enzymes which are often used for removing acnes. The cherry blossom has the effects of skin rejuvenation and brightening, is one of important raw materials. Therefore, the cherry blossom is known as the “flower of youth”.

(Centella asiatica)

The medicine properties of centella asiatica: bitter, pungent and cold, belonging to the liver, spleen, and kidney meridians. The traditional Chinese medicine centella asiatica contains various α-amyrin type triterpenoid components which contain racemic inositol, asiaticose, wax, carotenoids and chlorophyll, and the leaves contain β-glucose based quercetin and 3-glucose based kaempferol and 7-glucose based kaempferol. The centella asiatica has the effects of clearing heat, promoting dampness, detoxifying and reducing swelling. The centella asiatica contains various natural anti-inflammatory ingredients that can inhibit pathogenic bacteria and various viruses in the human body, thus having an auxiliary anti-inflammatory effect.

The centella asiatica has a wide range of effects. It is mainly used as an ointment for treating scars, can quickly protect the recovery of the skin and is conducive to enhancing the elasticity of the skin. It can also quickly eliminate swelling and pain, promote skin metabolism, accelerate skin growth, and alleviate dampness and heat in the body, promote blood circulation and stasis, and stop bleeding. For women, it can also alleviate the symptoms of menstrual pain during menstruation.

(Frankincense)

Frankincense is a resin exuded from the bark of a small arbor Ka frankincense tree in the olive family and its homologous plants. Frankincense is in the form of long oval shaped droplets, quasi circular particles, or irregular blocks of varying sizes, with a glassy or waxy luster on the broken surface. It has a pungent, bitter, and warm nature, and is beneficial to the heart, liver, and spleen meridians. It has the functions of promoting blood circulation, relieving pain, reducing swelling, and promoting muscle growth, as well as promoting menstrual flow.

1. Activating blood circulation and relieving pain: frankincense has a pungent, bitter, and warm taste, with fragrant qi. The pungent aroma can be diffused, the bitter can be broken, the warm energy can be unblocked, and the fragrance can flow through and enter the heart, liver, and spleen meridians, and the blood can be divided, so it can promote blood circulation and alleviate pain. It is commonly used to treat rheumatic pain, abdominal pain, and injuries caused by falls and other diseases.

2. Diminishing swelling and promoting muscle growth: frankincense has the effect of reducing swelling and promoting muscle growth, and can be used in clinical practice to treat carbuncle swelling and itching, improve skin related symptoms such as redness, swelling, heat, pain, and ulceration.

3. Tongjing: frankincense can also unblock and relieve pain, and is used to treat symptoms such as qi and blood stasis, dysmenorrhea, amenorrhea, postpartum blood stasis, and tingling.

(Patchouli)

The efficacy of patchouli: aroma dispels turbidity, stops vomiting, and releases heat relief. Patchouli is used for obstruction of dampness and turbidity, abdominal distension and vomiting, heat dampness surface syndrome, initial onset of dampness and temperature, fever and fatigue, chest tightness and discomfort, cold dampness and heat closure, abdominal pain, vomiting and diarrhea, nasal headache.

Patchouli has a bactericidal function, and one leaf in the mouth can eliminate bad breath, prevent infectious diseases, and can be used as a preservative. In summer, patchouli is used to cook congee or make tea to drink, which is effective for severe summer dampness, spleen and stomach dampness, abdominal distension, limb sleepiness, nausea and vomiti.

Patchouli itself is a plant with a fragrant aroma, and the entire plant has a fragrance, so patchouli is often combined with other plants with a fragrant aroma.

(Allium macrostemon)

The efficacy of allium macrostemon: taste: spicy, bitter, warm. Meridian: meridian of the heart, lungs, stomach, and large intestine. Contraindication: use with caution for those with weak stomach due to qi deficiency.

Allium macrostemon is capable of regulating Qi and broadening chest; clearing the yang and dispersing the knots. Master chest pain and heartache; chest and abdominal distension; coughing, wheezing, and phlegm accumulation; abdominal pain; Severe diarrhea; leucorrhea; Sores, furuncles, and carbuncle.

The antibacterial effect: allium macrostemon decoction has inhibitory effects on dysentery bacteria and Staphylococcus aureus. 300% allium macrostemon decoction was diluted in a test tube. It has an inhibitory effect on Staphylococcus aureus and pneumococcus in 1:4; it has an inhibitory effect on sarcina in 1:16.

The traditional Chinese medicine bag provided by the present disclosure consists of the above traditional Chinese medicines which are respectively calendula, cherry blossom, frankincense, patchouli, centella asiatica and allium macrostemon, belonging to aroma plants, and the combined breath can effectively improve the foul odor of crocodile oil and enhance its original efficacy, such as antibacterial, antiviral, anti-inflammatory, comprehensive regulation of skin activation cell regeneration, enhancement of skin immune function, efficient penetration, multi-layer energy supply, comprehensive balanced nutrition, moisturizing and water locking, firming and wrinkle removing, whitening and lightening, scar repair and anti-aging, etc.

In one embodiment of the present disclosure, a mass ratio of the calendula to the cherry blossom to the frankincense to the patchouli to the centella asiatica to the allium macrostemon is 1:1:1:1:1:1.

In one embodiment of the present disclosure, a mass ratio of the crocodile fat to the traditional Chinese medicine bag is (15-25 kg): (30-40 g). Since 1 kg=1000 g, after converted to the same unit, the mass ratio of them is (1500-2500):(3-4).

In a specific embodiment of the present disclosure, 6 g of calendula, 6 g of cherry blossom, 6 g of frankincense, 6 g of patchouli, 6 g of centella asiatica and 6 g of allium macrostemon, for 36 g in total, are put into a gauze bag to make the traditional Chinese medicine bag. 1 part of traditional Chinese medicine bag is added into every 20 kg of crocodile fat and then stands.

(2) Taking out the fat and then pulping to obtain a muddy material, filtering the muddy material, and then collecting filtrate to obtain crocodile oil.

In one embodiment of the present disclosure, pulping is carried out by using a pulping machine, the filtering is performed by using a filter mesh, and the pore diameter of the filter mesh is 150-300 meshes.

In one embodiment of the present disclosure, step (1) and step (2) are carried out at an ambient temperature of 15-25° C. In the present disclosure, there is no need to conduct extraction under the heating condition, because too high temperature can damage the active ingredients in crocodile fat.

In a specific embodiment of the present disclosure, the crocodile fat was successively washed and smashed after bloodiness and skins and fleshes therein are removed, followed by adding the traditional Chinese medicine bag and standing for 45-50 hours. The fat is separated from the medicine bag and then taken out, the taken fat is pulped to obtain a muddy material, and the muddy material is subjected to oil and residue separation. The ambient temperature of the above entire process is 15-25° C.

The present disclosure further relates to the crocodile oil extracted by the method, which is crystal and transparent, has no fishy smell, and can be stored for more than 3 years at 4-10° C.

### Example 1

An extraction method of natural crocodile oil comprises the following steps:

(1) 6 g of calendula, 6 g of cherry blossom, 6 g of frankincense, 6 g of patchouli, 6 g of centella asiatica and 6 g of allium macrostemon, for 36 g in total, were put into a gauze bag to make a traditional Chinese medicine bag.

(2) The crocodile fat was successively washed and smashed after bloodiness and skins and fleshes were removed, followed by adding 1 part of traditional Chinese medicine bag into every 20 kg of crocodile fat and then stood for 45-50 hours.

(3) The fat was separated from the medicine bag and then taken out, the taken fat was pulped to obtain a muddy material, the muddy material was subjected to oil and residue separation with a filter mesh whose pore diameter was 150-300 meshes. The filtrate was collected to obtain crocodile oil. The ambient temperature of the above entire process was 15-25° C.

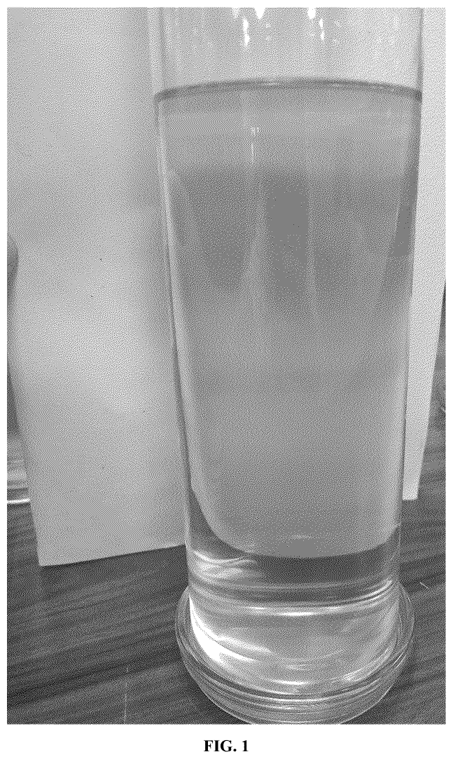

FIG. 1 is a picture of crocodile oil extracted in example 1 at room temperature of 20° C., from which it can be seen that crocodile oil is a transparent liquid in appearance, with good mobility and light yellow color.

Routine nine tests were performed on the crocodile oil obtained in example 1 in accordance with Safety Technical Specifications for Cosmetics (2015 Edition). The tests were entrusted to Guangdong Jianxun Testing Technology Co., Ltd. with report number 2233963-1. The results are seen in Table 1, indicating that the crocodile oil contains no heavy metals and harmful flora.

TABLE 1

| | | | Detection | | Detection | Index | Single |
|---|---|---|---|---|---|---|---|
| Number | Detection item | Detection method | results | Unit | limit | requirement | conclusion |
| 1 | Mercury | Safety Technical | <0.001 | mg/kg | 0.001 | ≤1 | Qualified |
| | | Specifications for | | | | | |
| | | Cosmetics (2015 | | | | | |
| | | Edition) Chapter | | | | | |
| | | 41.6 | | | | | |
| 2 | Lead | Safety Technical | 0.093 | mg/kg | 0.030 | ≤10 | Qualified |
| | | Specifications for | | | | | |
| | | Cosmetics (2015 | | | | | |
| | | Edition) Chapter | | | | | |
| | | 41.6 | | | | | |
| 3 | Arsenic | Safety Technical | 0.026 | mg/kg | 0.001 | ≤2 | Qualified |
| | | Specifications for | | | | | |
| | | Cosmetics (2015 | | | | | |
| | | Edition) Chapter | | | | | |
| | | 41.6 | | | | | |
| 4 | Cadmium | Safety Technical | Detectable, | mg/kg | 0.001 | ≤5 | Qualified |
| | | Specifications for | <0.0033 | | | | |
| | | Cosmetics (2015 | (quantitative | | | | |
| | | Edition) Chapter | concentration) | | | | |
| | | 41.6 | | | | | |
| 5 | Colony | Safety Technical | <10 | CFU/g | / | ≤1000 | Qualified |
| | forming unit | Specifications for | | | | | |
| | | Cosmetics (2015 | | | | | |
| | | Edition) Chapter | | | | | |
| | | 52 | | | | | |
| 6 | Thermotolerant | Safety Technical | Not | /g | /- | Not | Qualified |
| | coliforms | Specifications for | detectable | | | detectable | |
| | | Cosmetics (2015 | | | | | |
| | | Edition) Chapter | | | | | |
| | | 53 | | | | | |
| 7 | Pseudomonas | Safety Technical | Not | /g | / | Not | Qualified |
| | aeruginosa | Specifications for | detectable | | | detectable | |
| | | Cosmetics (2015 | | | | | |
| | | Edition) Chapter | | | | | |
| | | 54 | | | | | |
| 8 | Staphylococcus | Safety Technical | Not | /g | / | Not | Qualified |
| | aureus | Specifications for | detectable | | | detectable | |
| | | Cosmetics (2015 | | | | | |
| | | Edition) Chapter | | | | | |
| | | 55 | | | | | |
| 9 | Total number | Safety Technical | <10 | /g | / | ≤100 | Qualified |
| | of molds and | Specifications for | | | | | |
| | yeast | Cosmetics (2015 | | | | | |
| | | Edition) Chapter | | | | | |
| | | 56 | | | | | |

Energy and carbohydrate contents in crocodile oil obtained in example 1 were tested according to GB 28050-2011 (National Food Safety Standard—General Principles for Nutritional Labeling of Prepackaged Food). The tests were entrusted to Guangzhou Dongfang Zongheng Testing Service Co., Ltd. with report number RF22050140-001-T01S01. The results are seen in Table 2, indicating that the crocodile oil has high contents of trace elements, linolenic acid, linoleic acid, saturated fatty acid and unsaturated fat acid, and is suitable for external and oral administration.

TABLE 2

| | | | | Detection | |
|---|---|---|---|---|---|
| | | | | limit/quantitation | Detection |
| Number | Test item | Detection method | Unit | limit | results |
| 1 | Energy | Refer to GB/Z 21922-2008 | kJ/100 mL | — | 3696 |
| 2 | Protein | Refer to GB 5009.5-2016 first | g/100 mL | 0.008 g/100 mL | Not |
| | | law | | (Detection limit) | detectable |
| 3 | Fat | Refer to GB 5009.5-2016 | g/100 mL | 0.1 g/100 mL | 99.9 |
| | | second law | | (Quantitation limit) | |
| 4 | Carbohydrate | Refer to GB/Z 21922-2008 | g/100 mL | — | 0.1 |
| 5 | Sodium | Refer to GB 5009.91-2017 | mg/100 mL | 3 mg/100 mL | Not |
| | | first law | | (Quantitation limit) | detectable |
| 6 | Calcium | Refer to GB 5009.92-2016 | mg/100 mL | 0.15 mg/100 mL | 0.55 |
| | | first law | | (Quantitation limit) | |
| 7 | Iron | Refer to GB 5009.90-2016 | mg/100 mL | 0.25 mg/100 mL | Not |
| | | first law | | (Quantitation limit) | detectable |
| 8 | Zinc | Refer to GB 5009.14-2017 | mg/100 mL | 0.3 mg/100 mL | Not |
| | | first law | | (Quantitation limit) | detectable |
| 9 | Se | Refer to GB 5009.93-2017 | μg/100 mL | 0.6 μg/100 mL | 3.1 |
| | | first law | | (Quantitation limit) | |
| 10 | Cu | Refer to GB 5009.13-2017 | mg/100 mL | 0.05 mg/100 mL | Not |
| | | second law | | (Quantitation limit) | detectable |
| 11 | Linolenic acid | Refer to GB 5009.168-2016 | g/100 mL | 0.01 g/100 mL | 1.16 |
| | | first law | | (Quantitation limit) | |
| 12 | C20:5n3 cis | Refer to GB 5009.168-2016 | g/100 mL | 0.01 g/100 mL | 0.0242 |
| | 5,8,11,14,17-pentadecenoic | first law | | (Quantitation limit) | |
| | acid | | | | |
| 13 | Docosahexaenoic acid | Refer to GB 5009.168-2016 | g/100 mL | 0.01 g/100 mL | 0.466 |
| | | first law | | (Quantitation limit) | |
| 14 | Linoleic acid | Refer to GB 5009.168-2016 | g/100 mL | 0.01 g/100 mL | 20.1 |
| | | first law | | (Quantitation limit) | |
| 15 | Saturated fatty acid | Refer to GB 5009.168-2016 | g/100 mL | 0.01 g/100 mL | 27.3 |
| | | third law | | (Quantitation limit) | |
| 16 | Monounsaturated fatty acid | Refer to GB 5009.168-2016 | g/100 mL | 0.01 g/100 mL | 38.8 |
| | | third law | | (Quantitation limit) | |
| 17 | Unsaturated fatty acid | Refer to GB 5009.168-2016 | g/100 mL | 0.01 g/100 mL | 63.9 |
| | | third law | | (Quantitation limit) | |
| 18 | Polyunsaturated fatty acid | Refer to GB 5009.168-2016 | g/100 mL | 0.01 g/100 mL | 25.2 |
| | | third law | | (Quantitation limit) | |

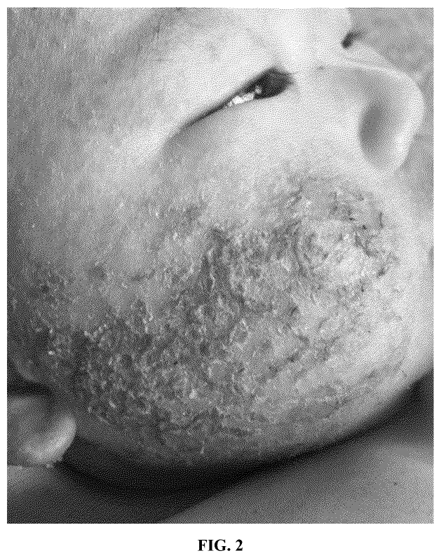

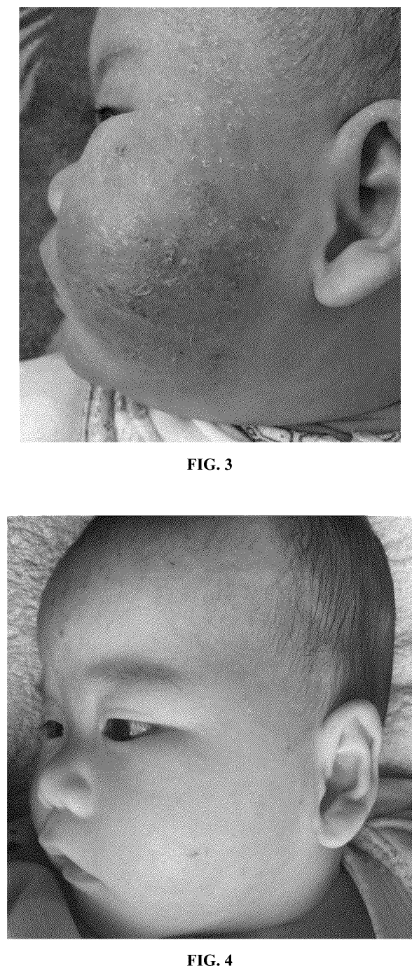

The crocodile oil extracted in example 1 is used for eczema treatment of 5-month-old infants. The eczema picture of infants before treatment is shown in FIG. 2, showing severe eczema on both cheeks and head, with deep red skin accompanied by surface scabs and dander. The use mode is applying the crocodile oil onto an affected part every three days. The eczema picture after one month is shown in FIG. 3, from which it can be obviously seen that the color of eczema becomes light red from dark red, and Facial scabs and dandruff are significantly improved. After further treatment for a halt month, the eczema is basically cured, and the face skin is smooth and has no scabs. The picture is shown in FIG. 4.

The above descriptions are only specific embodiments of the present disclosure, but the scope of protection of the present disclosure is not limited thereto. Within the technical scope disclosed in the present disclosure, any technicians familiar with the prior art can readily conceive that variations or replacements are all included within the scope of protection of the present disclosure. Therefore, the scope of protection of the present disclosure should be based on the scope of protection of appended claims.

What is claimed is:

1. An extraction method of natural crocodile oil, the method comprising the following steps:
(1) washing crocodile fat, smashing, adding the smashed crocodile fat into a traditional Chinese medicine bag, and then standing; and
(2) taking out the fat and then pulping to obtain a muddy material, filtering the muddy material, and then collecting filtrate to obtain crocodile oil.

2. The extraction method according to claim 1, wherein in step (1), the standing time is hours.

3. The extraction method according to claim 1, wherein in step (1), the traditional Chinese medicine bag comprises the following components: calendula, cherry blossom, frankincense, patchouli, centella asiatica, and allium macrostemon.

4. The extraction method according to claim 3, wherein in step (1), a mass ratio of the calendula to the cherry blossom to the frankincense to the patchouli to the centella asiatica to the allium macrostemon is 1:1:1:1:1:1.

5. The extraction method according to claim 1, wherein in step (1), a mass ratio of the crocodile fat to the traditional Chinese medicine bag is (1500-2500): (3-4).

6. The extraction method according to claim 1, wherein in step (2), the filtering is carried out by using a filter mesh.

7. The extraction method according to claim 6, wherein in step (2), the pore diameter of the filter mesh is 150-300 meshes.

8. The extraction method according to claim 1, wherein step (1) and step (2) are carried out at an ambient temperature of 15-25° C.

9. Crocodile oil obtained by the extraction method according to claim 1.
